SEQUENCE LISTING (3 sequences)

SEQ ID NO: 1 - protein, 486 aa, Geobacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFQFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTT

SEQ ID NO: 2 - protein, 686 aa, Geobacillus stearothermophilus
SSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 3 - protein, 599 aa, Trichorderma reesei
SVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

(19) United States
(12) Patent Application Publication
(10) Pub. No.: US 2022/0259528 A1
(43) Pub. Date: Aug. 18, 2022

(54) ENZYMES FOR INFUSION MASHING IN ADJUNCT BREWING TECHNICAL FIELD

(71) Applicant: DUPONT NUTRITION BIOSCIENCES APS, COPENHAGEN K (DK)

(72) Inventors: Jacob Flyvholm CRAMER, Palo Alto, CA (US); Trove BLADT, Skanderborg (DK)

(21) Appl. No.: 17/287,619

(22) PCT Filed: Oct. 21, 2019
(86) PCT No.: PCT/US19/57150

§ 371 Date: Apr. 22, 2021

Related U.S. Application Data
(60) Provisional application No. 62/748,739, filed on Oct. 22, 2018.

(51) Int. Cl. C12C 7/047; C12N 9/26; C12N 9/34; C12C 5/00

(52) U.S. Cl. CPC C12C 7/047; C12N 9/2414; C12Y 302/01001; C12C 5/004; C12Y 302/01133; C12N 9/2428; C12Y 302/01003; C12N 9/2411

(57) ABSTRACT

The present invention relates to methods of mashing 100% adjunct grists. More specifically, the instant disclosure provides methods and compositions wherein an alpha-amylase in combination with an maltogenic alpha amylase and/or glucoamylse to make a non-malt wort composed by adjunct raw materials.

### TECHNICAL FIELD

The present invention relates to methods of mashing adjunct grists. More specifically, the instant disclosure provides methods and compositions wherein an alpha-amylase in combination with an maltogenic alpha amylase and/or glucoamylase are employed in brewing to provide a non-malt wort composed by adjunct raw materials.

### BACKGROUND

Brewing generally involves three steps: malting, mashing and fermentation. The main purpose of the malting step is to develop enzymes which have a subsequent role during the brewing process in starch and protein degradation. Though traditionally beer has been brewed from just barley malt, hops and water; malt is an expensive raw material because it requires superior quality grains, water for germination and energy for kilning. To lower the cost of raw materials, unmalted grains, also called adjuncts, such as maize, rice, cassava, wheat, barley, rye, oat, quinoa and sorghum, maybe included in the brewing process. Adjuncts are primarily used because they are readily available and provide fermentable carbohydrates at a lower cost than barley malt.

The use of adjuncts in brewing complicates the traditional brewing process. Typically, adjuncts must be processed separately in a ‘cereal cooker’ to liquefy the starch. Thus, while the use of adjunct reduces the overall cost of raw materials, it requires an additional investment in a cereal cooker as well as an additional cost for heating and processing of the adjunct to liberate the fermentable sugars. To lessen these additional costs, brewers have tended to use low adjunct ratios (i.e. the ratio of adjunct to malt).

There is a continuing need for methods by which adjuncts can be used in beer production without requiring the use of a cereal cooker.

### SUMMARY OF THE INVENTION

In accordance with an aspect of the present invention, a method of mashing for 100% adjunct brewing is presented having the steps of: a.) providing a grist comprising adjunct; and b.) contacting the grist with an alpha amylase and a maltogenic alpha amylase and/or a glucoamylase to make a wort.

Optionally, the alpha amylase has at least 70% sequence identity to SEQ ID NO: 1. Optionally, the alpha amylase has at least 80% sequence identity to SEQ ID NO: 1. Optionally, the alpha amylase has at least 90% sequence identity to SEQ ID NO: 1. Optionally, the alpha amylase has at least 95% sequence identity to SEQ ID NO: 1. Optionally, the alpha amylase is an enzyme having a sequence according to SEQ ID NO: 1.

Optionally, the maltogenic alpha amylase has at least 70% sequence identity to SEQ ID NO: 2. Optionally, the maltogenic alpha amylase has at least 80% sequence identity to SEQ ID NO: 2. Optionally, the maltogenic alpha amylase has at least 90% sequence identity to SEQ ID NO: 2. Optionally, the maltogenic alpha amylase has at least 95% sequence identity to SEQ ID NO: 2. Optionally, the maltogenic alpha amylase is an enzyme having a sequence according to SEQ ID NO: 2.

Optionally, the glucoamylase has at least 70% sequence identity to SEQ ID NO: 3. Optionally, the glucoamylase has at least 80% sequence identity to SEQ ID NO: 3. Optionally, the glucoamylase has at least 90% sequence identity to SEQ ID NO: 3. Optionally, the glucoamylase has at least 95% sequence identity to SEQ ID NO: 3. Optionally, the glucoamylase is an enzyme having a sequence according to SEQ ID NO: 3.

In an aspect of the present invention, the grist is contacted with an alpha amylase and a maltogenic alpha amylase. Optionally, the alpha amylase has at least 70% sequence identity to SEQ ID NO: 1 and the maltogenic alpha amylase has at least 70% sequence identity to SEQ ID NO: 2. Optionally, the alpha amylase has at least 80% sequence identity to SEQ ID NO: 1 and the maltogenic alpha amylase has at least 80% sequence identity to SEQ ID NO: 2. Optionally, the alpha amylase has at least 90% sequence identity to SEQ ID NO: 1 and the maltogenic alpha amylase has at least 90% sequence identity to SEQ ID NO: 2. Optionally, the alpha amylase has at least 95% sequence identity to SEQ ID NO: 1 and the maltogenic alpha amylase has at least 95% sequence identity to SEQ ID NO: 2. Optionally, the alpha amylase is an enzyme having a sequence according to SEQ ID NO: 1 and the maltogenic alpha amylase is an enzyme having a sequence according to SEQ ID NO: 2.

In another aspect of the present invention, the grist is contacted with an alpha amylase and a glucoamylase. Optionally, the alpha amylase has at least 70% sequence identity to SEQ ID NO: 1 and the glucoamylase has at least 70% sequence identity to SEQ ID NO: 3. Optionally, the alpha amylase has at least 80% sequence identity to SEQ ID NO: 1 and the glucoamylase has at least 80% sequence identity to SEQ ID NO: 3. Optionally, the alpha amylase has at least 90% sequence identity to SEQ ID NO: 1 and the glucoamylase has at least 90% sequence identity to SEQ ID NO: 3. Optionally, the alpha amylase has at least 95% sequence identity to SEQ ID NO: 1 and the glucoamylase has at least 95% sequence identity to SEQ ID NO: 3. Optionally, the alpha amylase is an enzyme having a sequence according to SEQ ID NO: 1 and the glucoamylase is an enzyme having a sequence according to SEQ ID NO: 3.

Optionally, the grist is selected from the group consisting of corn, rice, sorghum and cassava or a mixture thereof. Optionally, the grist is at least 10% sorghum. Optionally, the grist is at least 25% sorghum. Optionally, the grist is at least 50% sorghum. Optionally, the grist is at least 75% sorghum. Optionally, the grist is 100% sorghum.

In other aspects of the present invention, the grist is at least 10% corn. Optionally, the grist is at least 25% corn. Optionally, the grist is at least 50% corn. Optionally, the grist is at least 75% corn. Optionally, the grist is 100% corn.

In another aspect, the grist is at least 10% rice. Optionally, the grist is at least 25% rice. Optionally, the grist is at least 50% rice. Optionally, the grist is at least 75% rice. Optionally, the grist is 100% rice.

In other aspects, the grist is at least 10% cassava. Optionally, the grist is at least 25% cassava. Optionally, the grist is at least 50% cassava. Optionally, the grist is at least 75% cassava. Optionally, the grist is 100% cassava.

Optionally, the wort is converted to beer.

In an aspect of the present invention, a use is provided of an alpha amylase and a maltogenic alpha amylase and/or a glucoamylase in brewing.

In another aspect of the present invention, an enzyme composition having an alpha amylase and a maltogenic alpha amylase is provided.

In yet another aspect of the present invention, an enzyme composition having an alpha amylase and a glucoamylase is provided.

### BRIEF DESCRIPTION OF THE BIOLOGICAL SEQUENCES

SEQ ID NO: 1 sets forth the mature amino acid sequence of the alpha amylase variant from Geobacillus stearothermophilus, GsAA1.

SEQ ID NO: 2 sets forth the mature amino acid sequence of the maltogenic alpha amylase from Geobacillus stearothermophilus, GsAA2.

SEQ ID NO: 3 sets forth the mature amino acid sequence of the glucoamylase from Trichoderma reesei.

### DETAILED DESCRIPTION OF THE INVENTION

The practice of the present teachings will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); Gene Transfer and Expression: A Laboratory Manual (Kriegler, 1990), and The Alcohol Textbook (Ingledew et al., eds., Fifth Edition, 2009), and Essentials of Carbohydrate Chemistry and Biochemistry (Lindhorste, 2007).

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this invention. Any methods and materials similar or equivalent to those described herein can be used in the practice or testing of the present teachings.

Numeric ranges provided herein are inclusive of the numbers defining the range.

### Definitions:

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleotide change. However, note that a polynucleotide encoding a wild-type, parental, or reference polypeptide is not limited to a naturally-occurring polynucleotide, and encompasses any polynucleotide encoding the wild-type, parental, or reference polypeptide.

Reference to the wild-type polypeptide is understood to include the mature form of the polypeptide. A “mature” polypeptide or variant, thereof, is one in which a signal sequence is absent, for example, cleaved from an immature form of the polypeptide during or following expression of the polypeptide.

The term “variant,” with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The term “recombinant,” when used in reference to a subject cell, nucleic acid, protein or vector, indicates that the subject has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding an amylase is a recombinant vector.

The terms “recovered,” “isolated,” and “separated,” refer to a compound, protein (polypeptides), cell, nucleic acid, amino acid, or other specified material or component that is removed from at least one other material or component with which it is naturally associated as found in nature. An “isolated” polypeptides, thereof, includes, but is not limited to, a culture broth containing secreted polypeptide expressed in a heterologous host cell.

The term “amino acid sequence” is synonymous with the terms “polypeptide,” “protein,” and “peptide,” and are used interchangeably. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The term “nucleic acid” encompasses DNA, RNA, heteroduplexes, and synthetic molecules capable of encoding a polypeptide. Nucleic acids may be single stranded or double stranded and may have chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art.

A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest. The term “host cell” includes protoplasts created from cells.

The term “heterologous” with reference to a polynucleotide or protein refers to a polynucleotide or protein that does not naturally occur in a host cell.

The term “endogenous” with reference to a polynucleotide or protein refers to a polynucleotide or protein that occurs naturally in the host cell.

The term “expression” refers to the process by which a polypeptide is produced based on a nucleic acid sequence. The process includes both transcription and translation.

A “selective marker” or “selectable marker” refers to a gene capable of being expressed in a host to facilitate selection of host cells carrying the gene. Examples of selectable markers include but are not limited to antimicrobials (e.g., hygromycin, bleomycin, or chloramphenicol) and/or genes that confer a metabolic advantage, such as a nutritional advantage on the host cell.

A “vector” refers to a polynucleotide sequence designed to introduce nucleic acids into one or more cell types. Vectors include cloning vectors, expression vectors, shuttle vectors, plasmids, phage particles, cassettes and the like.

An “expression vector” refers to a DNA construct comprising a DNA sequence encoding a polypeptide of interest, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequences.

A “signal sequence” is a sequence of amino acids attached to the N-terminal portion of a protein, which facilitates the secretion of the protein outside the cell. The mature form of an extracellular protein lacks the signal sequence, which is cleaved off during the secretion process.

“Biologically active” refers to a sequence having a specified biological activity, such an enzymatic activity.

The term “specific activity” refers to the number of moles of substrate that can be converted to product by an enzyme or enzyme preparation per unit time under specific conditions. Specific activity is generally expressed as units (U)/mg of protein.

As used herein, “percent sequence identity” means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF.
   Deletions are counted as non-identical residues, compared to a reference sequence. Deletions occurring at either terminus are included. For example, a variant with five amino acid deletions of the C-terminus of the mature 617 residue polypeptide would have a percent sequence identity of 99% (612/617 identical residues×100, rounded to the nearest whole number) relative to the mature polypeptide. Such a variant would be encompassed by a variant having “at least 99% sequence identity” to a mature polypeptide.

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

The term “about” refers to ±5% to the referenced value.

### Additional Mutations

In some embodiments, the present amylases further include one or more mutations that provide a further performance or stability benefit. Exemplary performance benefits include but are not limited to increased thermal stability, increased storage stability, increased solubility, an altered pH profile, increased specific activity, modified substrate specificity, modified substrate binding, modified pH-dependent activity, modified pH-dependent stability, increased oxidative stability, and increased expression. In some cases, the performance benefit is realized at a relatively low temperature. In some cases, the performance benefit is realized at a relatively high temperature.

Furthermore, the present amylases may include any number of conservative amino acid substitutions. Exemplary conservative amino acid substitutions are listed in the following Table.

### Conservative Amino Acid Substitutions

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4- carboxylic acid, D- |
| | | or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

The reader will appreciate that some of the above mentioned conservative mutations can be produced by genetic manipulation, while others are produced by introducing synthetic amino acids into a polypeptide by genetic or other means.

The present amylases may be “precursor,” “immature,” or “full-length,” in which case they include a signal sequence, or “mature,” in which case they lack a signal sequence. Mature forms of the polypeptides are generally the most useful. Unless otherwise noted, the amino acid residue numbering used herein refers to the mature forms of the respective amylase polypeptides. The present amylase polypeptides may also be truncated to remove the N or C-termini, so long as the resulting polypeptides retain amylase activity.

The present amylases may be a “chimeric” or “hybrid” polypeptide, in that it includes at least a portion of a first amylase polypeptide, and at least a portion of a second amylase polypeptide. The present amylases may further include heterologous signal sequence, an epitope to allow tracking or purification, or the like. Exemplary heterologous signal sequences are from B. licheniformis amylase (LAT), B. subtilis (AmyE or AprE), and Streptomyces CelA.

### Production of Amylases

The present amylases can be produced in host cells, for example, by secretion or intracellular expression. A cultured cell material (e.g., a whole-cell broth) comprising an amylase can be obtained following secretion of the amylase into the cell medium. Optionally, the amylase can be isolated from the host cells, or even isolated from the cell broth, depending on the desired purity of the final amylase. A gene encoding a proline specific amylase can be cloned and expressed according to methods well known in the art. Suitable host cells include bacterial, fungal (including yeast and filamentous fungi), and plant cells (including algae). Particularly useful host cells include Aspergillus niger, Aspergillus oryzae or Trichoderma reesei. Other host cells include bacterial cells, e.g., Bacillus subtilis or B. licheniformis, as well as Streptomyces, and E. Coli.

The host cell further may express a nucleic acid encoding a homologous or heterologous amylase that is not the same species as the host cell, or one or more other enzymes. The amylase may be a variant amylase. Additionally, the host may express one or more accessory enzymes, proteins, peptides.

### Vectors

A DNA construct comprising a nucleic acid encoding an amylase can be constructed to be expressed in a host cell. Because of the well-known degeneracy in the genetic code, variant polynucleotides that encode an identical amino acid sequence can be designed and made with routine skill. It is also well-known in the art to optimize codon use for a particular host cell. Nucleic acids encoding an amylase can be incorporated into a vector. Vectors can be transferred to a host cell using well-known transformation techniques, such as those disclosed below.

The vector may be any vector that can be transformed into and replicated within a host cell. For example, a vector comprising a nucleic acid encoding an amylase can be transformed and replicated in a bacterial host cell as a means of propagating and amplifying the vector. The vector also may be transformed into an expression host, so that the encoding nucleic acids can be expressed as a functional amylase. Host cells that serve as expression hosts can include filamentous fungi, for example.

A nucleic acid encoding an amylase can be operably linked to a suitable promoter, which allows transcription in the host cell. The promoter may be any DNA sequence that shows transcriptional activity in the host cell of choice and may be derived from genes encoding proteins either homologous or heterologous to the host cell. Exemplary promoters for directing the transcription of the DNA sequence encoding an amylase, especially in a bacterial host, are the promoter of the lac operon of E. coli, the Streptomyces coelicolor agarase gene dagA or celA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase (amyQ), the promoters of the Bacillus subtilis xylA and xylB genes etc. For transcription in a fungal host, examples of useful promoters are those derived from the gene encoding Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. When a gene encoding an amylase expressed in a bacterial species such as E. coli, a suitable promoter can be selected, for example, from a bacteriophage promoter including a T7 promoter and a phage lambda promoter. Examples of suitable promoters for the expression in a yeast species include but are not limited to the Gal 1 and Gal 10 promoters of Saccharomyces cerevisiae and the Pichia pastoris AOX1 or AOX2 promoters. cbh1 is an endogenous, inducible promoter from T. reesei. See Liu et al. (2008) “Improved heterologous gene expression in Trichoderma reesei by cellobiohydrolase I gene (cbh1) promoter optimization,” Acta Biochim. Biophys. Sin (Shanghai) 40(2): 158-65.

The coding sequence can be operably linked to a signal sequence. The DNA encoding the signal sequence may be the DNA sequence naturally associated with the amylase gene to be expressed or from a different genus or species. A signal sequence and a promoter sequence comprising a DNA construct or vector can be introduced into a fungal host cell and can be derived from the same source. For example, the signal sequence is the cbh1 signal sequence that is operably linked to a cbh1 promoter.

An expression vector may also comprise a suitable transcription terminator and, in eukaryotes, polyadenylation sequences operably linked to the DNA sequence encoding a variant amylase. Termination and polyadenylation sequences may suitably be derived from the same sources as the promoter.

The vector may further comprise a DNA sequence enabling the vector to replicate in the host cell. Examples of such sequences are the origins of replication of plasmids pUC19, pACYC177, pUB110, pE194, pAMB1, and pIJ702.

The vector may also comprise a selectable marker, e.g., a gene the product of which complements a defect in the isolated host cell, such as the dal genes from B. subtilis or B. licheniformis, or a gene that confers antibiotic resistance such as, e.g., ampicillin, kanamycin, chloramphenicol, or tetracycline resistance. Furthermore, the vector may comprise Aspergillus selection markers such as amdS, argB, niaD and xxsC, a marker giving rise to hygromycin resistance, or the selection may be accomplished by co-transformation, such as known in the art. See e.g., International PCT Application WO 91/17243.

Intracellular expression may be advantageous in some respects, e.g., when using certain bacteria or fungi as host cells to produce large amounts of amylase for subsequent enrichment or purification. Extracellular secretion of amylase into the culture medium can also be used to make a cultured cell material comprising the isolated amylase.

The expression vector typically includes the components of a cloning vector, such as, for example, an element that permits autonomous replication of the vector in the selected host organism and one or more phenotypically detectable markers for selection purposes. The expression vector normally comprises control nucleotide sequences such as a promoter, operator, ribosome binding site, translation initiation signal and optionally, a repressor gene or one or more activator genes. Additionally, the expression vector may comprise a sequence coding for an amino acid sequence capable of targeting the amylase to a host cell organelle such as a peroxisome, or to a particular host cell compartment. Such a targeting sequence includes but is not limited to the sequence, SKL. For expression under the direction of control sequences, the nucleic acid sequence of the amylase is operably linked to the control sequences in proper manner with respect to expression.

The procedures used to ligate the DNA construct encoding an amylase, the promoter, terminator and other elements, respectively, and to insert them into suitable vectors containing the information necessary for replication, are well known to persons skilled in the art (see, e.g., Sambrook et al., MOLECULAR CLONING: A LABORATORY MANUAL, 2^{nd} ed., Cold Spring Harbor, 1989, and 3rd ed., 2001).

### Transformation and Culture of Host Cells

An isolated cell, either comprising a DNA construct or an expression vector, is advantageously used as a host cell in the recombinant production of an amylase. The cell may be transformed with the DNA construct encoding the enzyme, conveniently by integrating the DNA construct (in one or more copies) in the host chromosome. This integration is generally considered to be an advantage, as the DNA sequence is more likely to be stably maintained in the cell. Integration of the DNA constructs into the host chromosome may be performed according to conventional methods, e.g., by homologous or heterologous recombination. Alternatively, the cell may be transformed with an expression vector as described above in connection with the different types of host cells.

Examples of suitable bacterial host organisms are Gram positive bacterial species such as Bacillaceae including Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus lautus, Bacillus megaterium, and Bacillus thuringiensis; Streptomyces species such as Streptomyces murinus; lactic acid bacterial species including Lactococcus sp. such as Lactococcus lactis; Lactobacillus sp. including Lactobacillus reuteri; Leuconostoc sp.; Pediococcus sp.; and Streptococcus sp. Alternatively, strains of a Gram negative bacterial species belonging to Enterobacteriaceae including E. coli, or to Pseudomonadaceae can be selected as the host organism.

A suitable yeast host organism can be selected from the biotechnologically relevant yeasts species such as but not limited to yeast species such as Pichia sp., Hansenula sp., or Kluyveromyces, Yarrowinia, Schizosaccharomyces species or a species of Saccharomyces, including Saccharomyces cerevisiae or a species belonging to Schizosaccharomyces such as, for example, S. pombe species. A strain of the methylotrophic yeast species, Pichia pastoris, can be used as the host organism. Alternatively, the host organism can be a Hansenula species. Suitable host organisms among filamentous fungi include species of Aspergillus, e.g., Aspergillus niger, Aspergillus oryzae, Aspergillus tubigensis, Aspergillus awamori, or Aspergillus nidulans. Alternatively, strains of a Fusarium species, e.g., Fusarium oxysporum or of a Rhizomucor species such as Rhizomucor miehei can be used as the host organism. Other suitable strains include Thermomyces and Mucor species. In addition, Trichoderma sp. can be used as a host. A suitable procedure for transformation of Aspergillus host cells includes, for example, that described in EP 238023. An amylase expressed by a fungal host cell can be glycosylated, i.e., will comprise a glycosyl moiety. The glycosylation pattern can be the same or different as present in the wild-type amylase. The type and/or degree of glycosylation may impart changes in enzymatic and/or biochemical properties.

It is advantageous to delete genes from expression hosts, where the gene deficiency can be cured by the transformed expression vector. Known methods may be used to obtain a fungal host cell having one or more inactivated genes. Gene inactivation may be accomplished by complete or partial deletion, by insertional inactivation or by any other means that renders a gene nonfunctional for its intended purpose, such that the gene is prevented from expression of a functional protein. A gene from a Trichoderma sp. or other filamentous fungal host that has been cloned can be deleted, for example, cbh1, cbh2, egl1, and egl2 genes. Gene deletion may be accomplished by inserting a form of the desired gene to be inactivated into a plasmid by methods known in the art.

Introduction of a DNA construct or vector into a host cell includes techniques such as transformation; electroporation; nuclear microinjection; transduction; transfection, e.g., lipofection mediated and DEAE-Dextrin mediated transfection; incubation with calcium phosphate DNA precipitate; high velocity bombardment with DNA-coated microprojectiles; and protoplast fusion. General transformation techniques are known in the art. See, e.g., Sambrook et al. (2001), supra. The expression of heterologous protein in Trichoderma is described, for example, in U.S. Pat. No. 6,022,725. Reference is also made to Cao et al. (2000) Science 9:991-1001 for transformation of Aspergillus strains. Genetically stable transformants can be constructed with vector systems whereby the nucleic acid encoding an amylase is stably integrated into a host cell chromosome. Transformants are then selected and purified by known techniques.

### Expression

A method of producing an amylase may comprise cultivating a host cell as described above under conditions conducive to the production of the enzyme and recovering the enzyme from the cells and/or culture medium.

The medium used to cultivate the cells may be any conventional medium suitable for growing the host cell in question and obtaining expression of an amylase. Suitable media and media components are available from commercial suppliers or may be prepared according to published recipes (e.g., as described in catalogues of the American Type Culture Collection).

An enzyme secreted from the host cells can be used in a whole broth preparation. In the present methods, the preparation of a spent whole fermentation broth of a recombinant microorganism can be achieved using any cultivation method known in the art resulting in the expression of an amylase. Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the amylase to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

An enzyme secreted from the host cells may conveniently be recovered from the culture medium by well-known procedures, including separating the cells from the medium by centrifugation or filtration, and precipitating proteinaceous components of the medium by means of a salt such as ammonium sulfate, followed by the use of chromatographic procedures such as ion exchange chromatography, affinity chromatography, or the like.

The polynucleotide encoding an amylase in a vector can be operably linked to a control sequence that is capable of providing for the expression of the coding sequence by the host cell, i.e. the vector is an expression vector. The control sequences may be modified, for example by the addition of further transcriptional regulatory elements to make the level of transcription directed by the control sequences more responsive to transcriptional modulators. The control sequences may in particular comprise promoters.

Host cells may be cultured under suitable conditions that allow expression of an amylase. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or Sophorose. Polypeptides can also be produced recombinantly in an in vitro cell-free system, such as the TNT™ (Promega) rabbit reticulocyte system.

### Methods For Enriching and Purifying Amylases

Fermentation, separation, and concentration techniques are well known in the art and conventional methods can be used in order to prepare an amylase polypeptide-containing solution.

After fermentation, a fermentation broth is obtained, the microbial cells and various suspended solids, including residual raw fermentation materials, are removed by conventional separation techniques in order to obtain an amylase solution. Filtration, centrifugation, microfiltration, rotary vacuum drum filtration, ultrafiltration, centrifugation followed by ultra-filtration, extraction, or chromatography, or the like, are generally used.

It is desirable to concentrate an amylase polypeptide-containing solution in order to optimize recovery. Use of unconcentrated solutions requires increased incubation time in order to collect the enriched or purified enzyme precipitate.

The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary drum vacuum filtration and/or ultrafiltration.

### Preferred Embodiments of the Invention

In accordance with an aspect of the present invention, it has been discovered that an adjunct starch with high gelatinization temperature can be efficiently liquefied and saccharified with processing temperatures lower than traditionally used for such starch types by a combination of an alpha amylase and a maltogenic alpha amylase and/or a glucoamylase to make a fermentable wort as set forth and claimed herein. Thus, an adjunct such as corn grist, corn starch, rice starch, sorghum starch or cassava among others starch sources can be processed without endogenous malt enzymes and (preferably) without prior gelatinization in a so-called infusion process. Liquefaction and saccharification of such adjunct starches requires that the mash is supplemented by an exogenously supplied enzyme composition. These starch adjuncts are normally characterized by a high gelatinization temperature, including a high onset gelatinization temperature. Surprisingly the right combination of enzymes may enable a high degree of starch solubilization/liquefaction and saccharification of said starch material, such the starch extracted during the process with increasing temperature is gradually hydrolyzed into fermentable sugars and smaller dextrins. Preferably the final mash is starch negative to iodine testing also correlating with a high extract value in the resulting wort. To achieve appropriate conversion of said sugars into ethanol by yeast fermentation, the fraction of DP4+ dextrins should preferable be less than 30% of the total sum of soluble sugar or even more preferable less than 25% of the total sum of soluble sugars. The mashing is finalized by mashing-off at a temperature of 70° C. or more; preferable at least 80° C.

SEQ ID NO: 1 sets forth the mature amino acid sequence of the alpha amylase variant from Geobacillus stearothermophilus, GsAA1.

SEQ ID NO: 2 sets forth the mature amino acid sequence of the maltogenic alpha amylase from Geobacillus stearothermophilus, GsAA2.

SEQ ID NO: 3 sets forth the mature amino acid sequence of the glucoamylase from Trichoderma reesei.

| SEQ | | |
|---|---|---|
| ID | | |
| NO.: | Sequence | Origin |
| 1 | AAPFNGTMMQYFEWYLPDDGTLWTKVANEANNLSSLGITALWLP | Geobacillus |
| | PAYKGTSRSDVGYGVYDLYDLGEFNQKGTVRTKYGTKAQYLQAI | stearothermophilus |
| | QAAHAAGMQVYADVVFDHKGGADGTEWVDAVEVNPSDRNQEIS | |
| | GTYQIQAWTKFDFPGRGNTYSSFKWRWYHFDGVDWDESRKLSRI | |
| | YKFRGIGKAWDWEVDTENGNYDYLMYADLDMDHPEVVTELKN | |
| | WGKWYVNTTNIDGFRLDAVKHIKFQFFPDWLSYVRSQTGKPLFTV | |
| | GEYWSYDINKLHNYITKTNGTMSLFDAPLHNKFYTASKSGGAFDM | |
| | RTLMTNTLMKDQPTLAVTFVDNHDTEPGQALQSWVDPWFKPLAY | |
| | AFILTRQEGYPCVFYGDYYGIPQYNIPSLKSKIDPLLIARRDYAYGT | |
| | QHDYLDHSDIIGWTREGVTEKPGSGLAALITDGPGGSKWMYVGKQ | |
| | HAGKVFYDLTGNRSDTVTINSDGWGEFKVNGGSVSVWVPRKTT | |
| 2 | SSSASVKGDVIYQIIIDRFYDGDTTNNNPAKSYGLYDPTKSKWKMY | Geobacillus |
| | WGGDLEGVRQKLPYLKQLGVTTIWLSPVLDNLDTLAGTDNTGYH | stearothermophilus |
| | GYWTRDFKQIEEHFGNWTTFDTLVNDAHQNGIKVIVDFVPNHSTPF | |
| | KANDSTFAEGGALYNNGTYWIGNYFDDATKGYFHHNGDISNWDD | |
| | RYEAQWKNFTDPAGFSLADLSQENGTIAQYLTDAAVQLVAHGAD | |
| | GLRIDAVKHFNSGFSKSLADKLYQKKDIFLVGEWYGDDPGTANHL | |
| | EKVRYANNSGVNVLDFDLNTVIRNVFGTFTQTMYDLNNMVNQTG | |
| | NEYKYKENLITFIDNHDMSRFLSVNSNKANLHQALAFILTSRGTPSI | |
| | YYGTEQYMAGGNDPYNRGMMPAFDTTTTAFKEVSTLAGLRRNNA | |
| | AIQYGTTTQRWINNDVYIYERKFFNDVVLVAINRNTQSSYSISGLQT | |
| | ALPNGSYADYLSGLLGGNGISVSNGSVASFTLAPGAVSVWQYSTS | |
| | ASAPQIGSVAPNMGIPGNVVTIDGKGFGTTQGTVTFGGVTATVKS | |
| | WTSNRIEVYVPNMAAGLTDVKVTAGGVSSNLYSYNILSGTQTSVV | |
| | FTVKSAPPTNLGDKIYLTGNIPELGNWSTDTSGAVNNAQGPLLAPN | |
| | YPDWFYVFSVPAGKTIQFKFFIKRADGTIQWENGSNHVATTPTGAT | |
| | GNITVTWQN | |
| 3 | SVDDFISTETPIALNNLLCNVGPDGCRAFGTSAGAVIASPSTIDPDY | Trichoderma reesei |
| | YYMWTRDSALVFKNLIDRFTETYDAGLQRRIEQYITAQVTLQGLSN | |
| | PSGSLADGSGLGEPKFELTLKPFTGNWGRPQRDGPALRAIALIGYS | |
| | KWLINNNYQSTVSNVIWPIVRNDLNYVAQYWNQTGFDLWEEVNG | |
| | SSFFTVANQHRALVEGATLAATLGQSGSAYSSVAPQVLCFLQRFW | |
| | VSSGGYVDSNINTNEGRTGKDVNSVLTSIHTFDPNLGCDAGTFQPC | |
| | SDKALSNLKVVVDSFRSIYGVNKGIPAGAAVAIGRYAEDVYYNGN | |
| | PWYLATFAAAEQLYDAIYVWKKTGSITVTATSLAFFQELVPGVTA | |
| | GTYSSSSSTFTNIINAVSTYADGFLSEAAKYVPADGSLAEQFDRNSG | |
| | TPLSALHLTWSYASFLTATARRAGIVPPSWANSSASTIPSTCSGASV | |
| | VGSYSRPTATSFPPSQTPKPGVPSGTPYTPLPCATPTSVAVTFHELVS | |
| | TQFGQTVKVAGNAAALGNWSTSAAVALDAVNYADNHPLWIGTV | |
| | NLEAGDVVEYKYINVGQDGSVTWESDPNHTYTVPAVACVTQVVK | |
| | EDTWQS | |

In accordance with an aspect of the present invention, a method of mashing for 100% adjunct brewing is presented having the steps of: a.) providing a grist comprising adjunct; and b.) contacting the grist with an alpha amylase and a maltogenic alpha amylase and/or a glucoamylase to make a wort.

Preferably, the alpha amylase has at least 70% sequence identity to SEQ ID NO: 1. More preferably, the alpha amylase has at least 80% sequence identity to SEQ ID NO: 1. Still more preferably, the alpha amylase has at least 90% sequence identity to SEQ ID NO: 1. In yet more preferred aspects, the alpha amylase has at least 95% sequence identity to SEQ ID NO: 1. In the most preferred aspects, the alpha amylase is an enzyme having a sequence according to SEQ ID NO: 1.

Preferably, the maltogenic alpha amylase has at least 70% sequence identity to SEQ ID NO: 2. More preferably, the maltogenic alpha amylase has at least 80% sequence identity to SEQ ID NO: 2. Still more preferably, the maltogenic alpha amylase has at least 90% sequence identity to SEQ ID NO: 2. In yet more preferred embodiments, the maltogenic alpha amylase has at least 95% sequence identity to SEQ ID NO: 2. In the most preferred embodiments, the maltogenic alpha amylase is an enzyme having a sequence according to SEQ ID NO: 2.

Preferably, the glucoamylase has at least 70% sequence identity to SEQ ID NO: 3. More preferably, the glucoamylase has at least 80% sequence identity to SEQ ID NO: 3. Still more preferably, the glucoamylase has at least 90% sequence identity to SEQ ID NO: 3. In yet more preferred embodiments, the glucoamylase has at least 95% sequence identity to SEQ ID NO: 3. In the most preferred embodiments, the glucoamylase is an enzyme having a sequence according to SEQ ID NO: 3.

In a preferred aspect of the present invention, the grist is contacted with an alpha amylase and a maltogenic alpha amylase. Preferably, the alpha amylase has at least 70% sequence identity to SEQ ID NO: 1 and the maltogenic alpha amylase has at least 70% sequence identity to SEQ ID NO: 2. More preferably the alpha amylase has at least 80% sequence identity to SEQ ID NO: 1 and the maltogenic alpha amylase has at least 80% sequence identity to SEQ ID NO: 2. Still more preferably, the alpha amylase has at least 90% sequence identity to SEQ ID NO: 1 and the maltogenic alpha amylase has at least 90% sequence identity to SEQ ID NO: 2. In yet more preferred embodiments the alpha amylase has at least 95% sequence identity to SEQ ID NO: 1 and the maltogenic alpha amylase has at least 95% sequence identity to SEQ ID NO: 2. In the most preferred embodiments, the alpha amylase is an enzyme having a sequence according to SEQ ID NO: 1 and the maltogenic alpha amylase is an enzyme having a sequence according to SEQ ID NO: 2.

In another preferred embodiment of the present invention, the grist is contacted with an alpha amylase and a glucoamylase. Preferably, the alpha amylase has at least 70% sequence identity to SEQ ID NO: 1 and the glucoamylase has at least 70% sequence identity to SEQ ID NO: 3. More preferably, the alpha amylase has at least 80% sequence identity to SEQ ID NO: 1 and the glucoamylase has at least 80% sequence identity to SEQ ID NO: 3. Still more preferably, the alpha amylase has at least 90% sequence identity to SEQ ID NO: 1 and the glucoamylase has at least 90% sequence identity to SEQ ID NO: 3. In yet more preferred embodiments, the alpha amylase has at least 95% sequence identity to SEQ ID NO: 1 and the glucoamylase has at least 95% sequence identity to SEQ ID NO: 3. In the most preferred embodiments, the alpha amylase is an enzyme having a sequence according to SEQ ID NO: 1 and the glucoamylase is an enzyme having a sequence according to SEQ ID NO: 3.

Preferably, the grist is selected from the group consisting of corn, rice, sorghum and cassava or a mixture thereof. More preferably, the grist is at least 10% sorghum. More preferably, the grist is at least 25% sorghum. Still more preferably, the grist is at least 50% sorghum. In yet more preferred embodiments the grist is at least 75% sorghum. In the most preferred embodiments the grist is 100% sorghum.

In other preferred embodiments, the grist is at least 10% corn. More preferably, the grist is at least 25% corn. Still more preferably, the grist is at least 50% corn. In yet more preferred embodiments the grist is at least 75% corn. In the most preferred embodiments the grist is 100% corn.

In other preferred embodiments, the grist is at least 10% rice. More preferably, the grist is at least 25% rice. Still more preferably, the grist is at least 50% rice. In yet more preferred embodiments the grist is at least 75% rice. In the most preferred embodiments the grist is 100% rice.

In other preferred embodiments, the grist is at least 10% cassava. More preferably, the grist is at least 25% cassava. Still more preferably, the grist is at least 50% cassava. In yet more preferred embodiments the grist is at least 75% cassava. In the most preferred embodiments the grist is 100% cassava.

Preferably, the wort is converted to beer.

In an aspect of the present invention, a use is provided of an alpha amylase and a maltogenic alpha amylase and/or a glucoamylase in brewing.

In another aspect of the present invention, an enzyme composition having an alpha amylase and a maltogenic alpha amylase is provided.

In yet another aspect of the present invention, an enzyme composition having an alpha amylase and a glucoamylase is provided.

The following examples are offered to illustrate, but not to limit the claimed disclosure.

### EXAMPLES

### Example 1—Enzymes

GsAA1: An alpha amylase variant from Geobacillus stearothermophilus having the amino acid sequence shown in SEQ ID NO:1
GsAA2: A maltogenic alpha amylase from Geobacillus stearothermophilus having the amino acid sequence shown in SEQ ID NO:2
TrGA: A glucoamylase from Trichoderma reesei having the amino acid sequence shown in SEQ ID NO:3
As an example of an alpha-amylase, AMYLEX® 5T (A 5T) from DuPont, was used. As example of a maltogenic alpha-amylase, DIAZYME® MA (D MA) from DuPont, was used. As an example of a gluco-amylase, DIAZYME® TGA (D TGA) from DuPont, was used.

### Example 2—Protein Determination Methods

Protein Determination by Stain Free Imager Criterion

Protein was quantified by SDS-PAGE gel and densitometry using Gel Doc™ EZ imaging system. Reagents used in the assay: Concentrated (2×) Laemmli Sample Buffer (Bio-Rad, Catalogue #161-0737); 26-well XT 4-12% Bis-Tris Gel (Bio-Rad, Catalogue #345-0125); protein markers “Precision Plus Protein Standards” (Bio-Rad, Catalogue #161- 0363); protein standard BSA (Thermo Scientific, Catalogue #23208) and SimplyBlue Safestain (Invitrogen, Catalogue #LC 6060. The assay was carried out as follow: In a 96 well-PCR plate 50 μL diluted enzyme sample were mixed with 50 μL sample buffer containing 2.7 mg DTT. The plate was sealed by Microseal ‘B’ Film from Bio-Rad and was placed into PCR machine to be heated to 70° C. for 10 minutes. After that the chamber was filled by running buffer, gel cassette was set. Then 10 μL of each sample and standard (0.125-1.00 mg/mL BSA) was loaded on the gel and 5 μL of the markers were loaded. After that the electrophoresis was run at 200 V for 45 min. Following electrophoresis, the gel was rinsed 3 times 5 min in water, then stained in Safestain overnight and finally destained in water. Then the gel was transferred to Imager. Image Lab software was used for calculation of intensity of each band. A calibration curve was made using BSA (Thermo Scientific, Catalogue #23208). The amount of the target protein was determined by the band intensity and calibration curve. The protein quantification method was employed to prepare enzyme samples of used in subsequent Examples.

### Example 3—Sugar Analysis by HPLC

All standards: Glucose, Maltose, Maltotriose and Maltotetraose were prepared in double distilled water (ddH2O) and filtered through 0.45 μm syringe filters. A set of each standard was prepared ranging in concentration from 10 to 100,000 ppm.

All wort samples containing active enzymes were inactivated by heating the sample to 95° C. for 10 min. Subsequently wort samples were prepared in 96 well MTP plates (Corning, N.Y., USA) and diluted minimum 4 times in ddH2O and filtered through 0.20 μm 96 well plate filters before analysis (Corning filter plate, PVDF hydrophile membrane, NY, USA). All samples were analyzed in duplicates.

### Instrumentation

Quantification of sugars: DP1, DP2, DP3, DP4 and DP5+ were performed by UPLC. Analysis of samples was carried out on a Dionex Ultimate 3000 UPLC system (Thermo Fisher Scientific) equipped with a DGP-3600SD Dual-Gradient analytical pump, WPS-3000TSL thermostated autosampler, TCC-3000SD thermostated column oven, and a RI-101 refractive index detector (Shodex, JM Science). Chromeleon datasystem software (Version 6.80, DU10A Build 2826, 171948) was used for data acquisition and analysis.

### Chromatographic Conditions

The samples were analyzed using an RSO oligosaccharide column, Ag⁺ 4% crosslinked (Phenomenex, The Netherlands) equipped with an analytical guard column (Carbo-Ag⁺ neutral, AJ0-4491, Phenomenex, The Netherlands) operated at 70° C. The column was eluted with double distilled water (filtered through a regenerated cellulose membrane of 0.45 μm and purged with helium gas) at a flow rate of 0.3 ml/min. Isocratic flow of 0.3 ml/min was maintained throughout analysis with a total run time of 45 min and injection volume was set to 10 μL. Samples were held at 20° C. in the thermostated autosampler compartment. The eluent was monitored by means of a refractive index detector (RI-101, Shodex, JM Science) and quantification was made by the peak area relative to the peak area of the given standard (DP1: glucose; DP2: maltose; DP3: maltotriose and peaks with a degree of four or higher maltotetraose was used as standard).

### Example 4—Low Temperature Infusion Mashing With Corn, Rice, Sorghum and Cassava Using Enzymes to Enable Extract and Fermentable Sugar

The objective of this example was to demonstrate the benefit (fermentable sugar and extract released) of having two enzymes present (maltogenic alpha-amylase or glucoamylase and an alpha amylase) during processing of adjunct in an infusion process (single vessel), compared to only having one of the enzymes to liberate the fermentable sugars.

Enzymes was tested in a mashing operation model system for wort production using corn grist (Nordgetreide GmBH Lubec, Germany), rice grist (Cambodia. MEKONG Asian Market, Dagrofa Brabrand), Sorghum (Sorghum, white, not grounded—Diageo, Ireland) and Cassava flour (Uganda) and a fixed water to grist ratio of 4:1. Rice grist was milled at a Buhler Miag malt mill 0.5 mm setting and sorghum was milled at setting 1.6 mm.

### Mashing Operation For Wort Production

Maize grits (3.0 g), Rice grist (milled 3.0 g), Sorghum (3.0 g) or Cassava flour (3.0 g) was mixed in Wheaton cups (glass containers with cap) preincubated with 12.0 g tap water at 64° C., pH adjusted to pH 5.4 with 2.5M sulphuric acid. Enzymes were added based on mg protein (in total 0.5 mL) determined according to example 2 and water as no enzyme control. Beside addition of GsAA1, GsAA2 and TrGA a fixed concentration of 0.5 mg/g grist Laminex® 750 (Dupont) were used to ensure filterability (B-glucanase) (this has no effect on the release of fermentable sugars). The Wheaton cups were placed in Drybath (Thermo Scientific Stem station) with magnetic stirring and the following mashing program was applied; samples were held at 64° C. for 60 minutes; heated to 80° C. for 10 minutes; and finally kept at 80° C. for 55 minutes mashed off. 15 ml sample was transferred to Falcon tubes and spent grains was separated from the wort by centrifugation in a Heraeus Multifuge X3R at 4500 rpm for 20 minutes at 10° C. The extract was measured by a handheld Plato Refractometer (PAL-PLATO, Atago, Tokyo). All samples were diluted 10× in H₂O and boiled in waterbath for 20 minutes to inactivate enzymes. Supernatant was collected and filtered (0.2 μm) for HPLC sugar analysis, as described in example 3.

The results using corn, rice, sorghum and cassava are shown in table 1 to 4 respectively. The addition of an endo-acting alpha amylase was observed to increase the extract of the wort compared to either no enzyme used that for all adjunct types were not processable (n.p.) or significantly lower using maltogenic alpha amylase or glucoamylase alone. The amount of non-fermentable sugar left in the wort was reduced in the current example by the addition of TrGA, GsAA2 and GsAA1 in the given order. Thus, largest reduction in non-fermentable sugars (DP4+) were obtained by GsAA2 and TrGA and for some adjuncts an additive effect was seen of these individually with together GsAA1. In conclusion, an infusion wort with high extract (>15°P) and high degree of fermentable sugar (<30% DP4+) was independently of raw material type (corn, rice, sorghum and cassava) produced by addition of an endo-acting alpha-amylase (GsAA1) and a maltogenic alpha-amylase (GsAA2) or an endo-acting alpha-amylase (GsAA1) and a glucoamylase (TrGA).

The results using blends of the raw materials (50% Sorghum and 50% Corn; 50% Sorghum and 50% Rice; 50% Rice and 50% Corn) are shown in table 6. A similar effect of the enzymes was observed regarding achieving high extract (>15°P) and high degree of fermentable sugar (<30% DP4+), compared to producing the wort from the individual raw materials in in-fusion mashing.

TABLE 1
100% Corn adjunct. Wort non-fermentable sugars
(DP4+ % of total sugars) and extract of wort in
degree Plato. Dosage of alpha amylase (GsAA1),
maltogenic alpha amylase (GsAA2) and glucoamylase
(TrGA) given as amount protein mg/g DM grist.
100% Corn

| Enzyme Dosage (mg/g corn) | | | | |
|---|---|---|---|---|
| Alpha | Maltogenic | | | |
| amylase | alpha amylase | Glucoamylase | | Extract |
| (GsAA1) | (GsAA2) | (TrGA) | % DP4+ | °Plato |
| — | — | — | 96.4 | n.p. |
| 0.025 | — | — | 64.0 | 16.0 |
| — | 0.02825 | — | 42.8 | 12.9 |
| — | — | 0.0865 | 21.2 | 6.7 |
| 0.025 | 0.02825 | — | 25.1 | 15.8 |
| 0.025 | — | 0.0865 | 26.3 | 15.7 |

TABLE 2
100% Rice adjunct. Wort non-fermentable sugars
(DP4+ % of total sugars) and extract of wort in
degree Plato. Dosage of alpha amylase (GsAA1),
maltogenic alpha amylase (GsAA2) and glucoamylase
(TrGA) given as amount protein mg/g DM grist.
100% Rice

| Enzyme Dosage (mg/g rice) | | | | |
|---|---|---|---|---|
| Alpha | Maltogenic | | | |
| amylase | alpha amylase | Glucoamylase | | Extract |
| (GsAA1) | (GsAA2) | (TrGA) | % DP4+ | °Plato |
| — | — | — | n.p. | n.p. |
| 0.025 | — | — | 49.1 | 16.0 |
| — | 0.02825 | — | 37.5 | 11.2 |
| — | — | 0.0865 | 23.1 | n.p. |
| 0.025 | 0.02825 | — | 18.3 | 16.7 |
| 0.025 | — | 0.0865 | 29.1 | 16.5 |

TABLE 3
100% sorghum. Wort non-fermentable sugars
(DP4+ % of total sugars) and extract of wort in
degree Plato. Dosage of alpha amylase (GsAA1),
maltogenic alpha amylase (GsAA2) and glucoamylase
(TrGA) given as amount protein mg/g DM grist.
100% Sorghum

| Enzyme Dosage (mg/g sorghum) | | | | |
|---|---|---|---|---|
| Alpha | Maltogenic | | | |
| amylase | alpha amylase | Glucoamylase | | Extract |
| (GsAA1) | (GsAA2) | (TrGA) | % DP4+ | °Plato |
| — | — | — | 93.2 | n.p. |
| 0.025 | — | — | 52.0 | 15.1 |
| — | 0.02825 | — | 44.2 | 13.4 |
| — | — | 0.0865 | 42.5 | n.p. |
| 0.025 | 0.02825 | — | 18.3 | 16.7 |
| 0.025 | — | 0.0865 | 29.1 | 16.5 |

TABLE 4
100% cassava. Wort non-fermentable sugars
(DP4+ % of total sugars) and extract of wort in
degree Plato. Dosage of alpha amylase (GsAA1),
maltogenic alpha amylase (GsAA2) and glucoamylase
(TrGA) given as amount protein mg/g DM grist.
100% Cassava

| Enzyme Dosage (mg/g cassava) | | | | |
|---|---|---|---|---|
| Alpha | Maltogenic | | | |
| amylase | alpha amylase | Glucoamylase | | Extract |
| (GsAA1) | (GsAA2) | (TrGA) | % DP4+ | °Plato |
| — | — | — | 88.6 | n.p. |
| 0.025 | — | — | 72.7 | 18.6 |
| — | 0.02825 | — | 61.8 | n.p. |
| — | — | 0.0865 | 23.7 | n.p. |
| 0.025 | 0.02825 | — | 18.3 | 18.9 |
| 0.025 | — | 0.0865 | 29.1 | 19.0 |

TABLE 5
50% sorghum - 50% corn and 50% sorghum -
50% rice and 50% rice - 50% corn. Wort non-
fermentable sugars (DP4+ % of total sugars) and
extract of wort in degree Plato. Dosage of Alpha
amylase (GsAA1), Maltogenic alpha amylase (GsAA2)
and Glucoamylase (TrGA) given as amount protein.

| Enzyme Dosage (mg/g adjunct) | | | | |
|---|---|---|---|---|
| Alpha | Maltogenic | | | |
| amylase | alpha amylase | Glucoamylase | | Extract |
| (GsAA1) | (GsAA2) | (TrGA) | % DP4+ | °Plato |

50% Sorghum and 50% Corn

| | | | | |
|---|---|---|---|---|
| 0.025 | 0.02825 | — | 21.7 | 16.4 |
| 0.025 | — | 0.0865 | 22.8 | 15.8 |

50% Sorghum and 50% Rice

| | | | | |
|---|---|---|---|---|
| 0.025 | 0.02825 | — | 17.0 | 16.0 |
| 0.025 | — | 0.0865 | 26.3 | 15.3 |

50% Rice and 50% Corn

| | | | | |
|---|---|---|---|---|
| 0.025 | 0.02825 | — | 18.0 | 16.3 |
| 0.025 | — | 0.0865 | 28.3 | 16.3 |

### Example 5—Application of Alpha-Amylase, Maltogenic Alpha-Amylase and Gluco-Amylase For Wort Production

### Lab Scale Infusion Mashing Operation With Filtration For Wort Production

Alpha-amylase, maltogenic alpha-amylase and gluco-amylase were tested in mashing operation with 100% Corn grits (Nordgetreide GmBH Lübec, Germany, Batch: 01.11.2016.), using a water to grist ratio of 3.8:1.

The corn adjunct was processed in the follow way: corn grits (70.0 g) and tap water (263 g) was mixed in mashing bath (Lockner, LG-electronics) cups and pH adjusted to pH 5.4 with 2.5M sulphuric acid. The corn adjunct was mashed with the program; heated to 63° C. and enzymes were applied; kept at 63° C. for 76 minutes for mashing in and saccharification; heated to 80° C. for 8.5 minutes by increasing temperature with 2° C./minute; kept at 80° C. for 35.5 minutes and mashing off.

At the end of mashing, the mashes were made up to 350 g with tap water and the content was separated into wort and spent corn. Wort volumes were measured after 30 minutes separation and were analyzed for extract and distribution of different solubilized sugar types measured as percentage of DP1, DP2, DP3 and DP4+.

One alpha-amylase (AA), one maltogenic alpha-amylase (MA) and one gluco-amylase (GA) was tested. As an example of an alpha-amylase, AMYLEX® 5T (A 5T) from DuPont, was used. As example of a maltogenic alpha-amylase, DIAZYME® MA (D MA) from DuPont, was used. As an example of a gluco-amylase, DIAZYME® TGA (D TGA) from DuPont, was used. The following dosages and combinations were tested: No enzymes; AMYLEX® 5T (2.5 kg/t of corn); DIAZYME® MA (1.5 kg/t of corn); AMYLEX® 5T (2.5 kg/t of corn) and DIAZYME® MA (1.5 kg/t of corn); DIAZYME® TGA (3.0 kg/t of corn); AMYLEX® 5T (2.5 kg/t of corn) and DIAZYME® TGA (3.0 kg/t of corn) as shown in Table 6.

TABLE 6
Concentration of enzyme product in kg/t of
corn used either as single or in combination
during mashing for wort production

| | Enzymes Applied at | | |
|---|---|---|---|
| | Mashing | | |
| No | A 5T | D MA | D TGA |
| enzymes | kg/t of corn | | |
| AA | 2.5 | | |
| MA | | 1.5 | |
| AA + MA | 2.5 | 1.5 | |
| GA | | | 3.0 |
| AA + GA | 2.5 | | 3.0 |

Wort analysis: The wort volume of each sample was measured after 30 minutes of mash separation following Dupont Standard Instruction Brewing, 23.8580-B11. In short, the sample was filtered through a plastic funnel with filter paper (VWR, European Cat. No. 516-0310, size 320 mm, folded qualitative filter paper, 307 Brewery grade, medium filtration rate) that was placed on top of a 250 ml measuring cylinder glass and time recorded. After 30 min, the amount of liquid that has passed through the filter (filtrate) into the measuring cylinder glass was measured. Original Extract (OE), extract in the wort samples after mashing was measured using Anton Paar (Lovis) following Dupont Standard Instruction Brewing, 23.8580-B28 (Based on EBC 8.3 Extract of Wort). Fermentable sugars (% total+g/100 mL) by HPLC were DP1, DP2, DP3 and DP4+ was determined after mashing following Dupont Standard Instruction Brewing, 23.8580-B20 (Based on EBC 8.7 Fermentable Carbohydrates in Wort by HPLC (IM)). The following: wort volumes, extracts and degree of fermentable sugars, expressed as the sum of % DP1 to DP3 released during mashing by applying the different enzymes as single or in combinations, is shown in Table 7.

TABLE 7
Wort volume, wort extract and relative
concentration in % of the sum of DP1 to DP3.

| | Volume of | | Sum of |
|---|---|---|---|
| | Wort (mL) after | Extract | % DP1 to |
| 100% CORN grits | 30 min separation | (°P) | DP3 |
| No enzyme | few droplets | n.a. | n.a. |
| 2.5 A 5T | 215 | 16.2 | 35.1 |
| 1.5 D MA | 42 | 13.1 | 56.6 |
| 2.5 A 5T + 1.5 D MA | 214 | 16.4 | 73.5 |
| 3.0 D TGA | few droplets | n.a. | n.a. |
| 2.5 A 5T + 3.0 D TGA | 213 | 16.5 | 79.4 |

As seen from the amount of wort achieved (Table 7) it was not possible to process 100% corn with the given mashing protocol without any application of exogenous enzymes nor if a gluco-amylase only was applied. To achieve acceptable amounts of wort and extract the alpha-amylase was required during mashing. However, if an alpha-amylase (endo-acting) only was applied the relative concentration of fermentable sugar types, expressed by the sum of DP1 to DP3 was too low for acceptable attenuation of any beer style. To achieve high percentage of DP1 to DP3 (fermentable sugars) the alpha-amylase needed to be combined with either a maltogenic alpha-amylase or a gluco-amylase enzyme.

### Example 6—Lab Scale Fermentability of Wort From Example 5

### Fermentation Operation For Attenuated Samples

The wort samples produced as described in example 7 that provided sufficient amount of wort for fermentation (>100 mL) were adjusted to pH 5.2 with 2.5 M sulphuric acid and one pellet of bitter hops from Hopfenveredlung, St. Johann: Alpha content of 16.0% (EBC 7.7 0 specific HPLC analysis, 01.10.2013), was added to each flask (in total 210 g). The wort samples were boiled for 60 minutes in a boiling bath and wort were cooled down to 17° C. and filtered. 100 g of each wort was weighted out into a 500 mL conical flask for fermentation adding 0.5% W34/70 (Weihenstephan) freshly produced yeast (0.50 g) to the wort having 17° C. The wort samples were fermented at 18° C. and 150 rpm after yeast addition. Analysis was performed when fermentation had finished.

Beer analysis: RDF was measured using an Anton Paar (DMA 5000) following Dupont Standard Instruction Brewing, 23.8580-B28 (Based on EBC 8.3 Extract of Wort) and alcohol by Dupont Standard Instruction Brewing, 23.8580-B28 (Based on EBC 8.3 Extract of Wort).
Real degree of fermentation (RDF) value may be calculated according to the equation below:

$$R D F ( \% ) = ( 1 - \frac{R E}{{}_{}^{o}{P}{}_{initial}^{}} ) \times 100$$

Where: RE=real extract=(0.1808×°Pᵢₙᵢₜᵢₐₗ)+(0.8192×°P_{final}), °Pᵢₙᵢₜᵢₐₗ is the specific gravity of the standardized worts before fermentation and °P_{final} is the specific gravity of the fermented worts expressed in degree Plato.
In the present context, Real degree of fermentation (RDF) was determined from the specific gravity and alcohol concentration.
Specific gravity and alcohol concentration was determined on the fermented samples using a Beer Alcolyzer Plus and a DMA 5000 Density meter (both from Anton Paar, Gratz, Austria). Based on these measurements, the real degree of fermentation (RDF) value was calculated according to the equation below:

$$R D F ( \% ) = \frac{O E - E ( r )}{O E} \times 100$$

Where: E(r) is the real extract in degree Plato (°P) and OE is the original extract in °P.
Original Extract (OE) Extract in the beer samples after mashing was measured using an Anton Paar (DMA 5000) following Dupont Standard Instruction Brewing, 23.8580-B28 (Based on EBC 8.3 Extract of Wort). Alcohol by volume (% V/V) The achieved Alcohol By Volume (ABV) was measured using an Anton Paar (DMA 5000) following Dupont Standard Instruction Brewing, 23.8580-B28 (Based on EBC 8.3 Extract of Wort).
The following Real Degree of Fermentation (RDF) after fermentation of wort having applied the alpha-amylase in combination with either a maltogenic alpha-amylase alone or a gluco-amylase during mashing is shown in Table 8.

TABLE 8
RDF after fermentation of wort produced
with an alpha-amylase either alone or in
combination with a maltogenic alpha-
amylase or a gluco-amylase.

| 100% CORN grits | % RDF |
|---|---|
| 2.5 A 5T | 29.3 |
| 2.5 A 5T + 1.5 D MA | 60.8 |
| 2.5 A 5T + 3.0 D TGA | 64.5 |

The obtained RDF values corresponded with the analysis of sugar composition in the applied wort, thus a relative higher content of DP1 to DP3 (fermentable) sugar in the wort lead to a higher % RDF value of the fermentation. The combination of alpha-amylase and gluco-amylase or alpha-amylase and maltogenic alpha-amylase showed increased RDF values compared to applying the alpha-amylase only. Hence, the alpha-amylase needed to be combined with either a maltogenic alpha-amylase or a gluco-amylase for achieving a satisfactory attenuation for some beer styles.

### Example 7—Application of the Combination of Alpha-Amylase, Maltogenic Alpha-Amylase and Gluco-Amylase For Wort Production

### Mashing Operation For Wort Production

Alpha-amylase, maltogenic alpha-amylase and gluco-amylase were all tested in mashing operation with 100% Corn grits (Nordgetreide GmBH Lubec, Germany, Batch: 01.11.2016.), using a water to grist ratio of 3.8:1.

The corn adjunct was processed in the follow way: corn grits (70.0 g) and tap water (263 g) was mixed in mashing bath (Lockner, LG-electronics) cups and pH adjusted to pH 5.4 with 2.5M sulphuric acid. The corn adjunct was mashed with the program; heated to 63° C. and enzymes were applied; kept at 63° C. for 60 minutes for mashing in and saccharification; heated to 75° C. for 8 minutes by increasing temperature with 1.5° C./minute; kept at 75° C. for 20 minutes;

heated to 80° C. for 3 minutes, 3 minutes by increasing temperature with 1.5 ° C./minute; kept at 80° C. for 20 minutes and then mashing off.

At the end of mashing, the mashes were made up to 350 g with tap water and the content was separated into wort and spent corn. Wort volumes were measured after 30 minutes separation and were analyzed for extract and distribution of different solubilized sugar types measured as percentage of DP1, DP2, DP3 and DP4+.

One alpha-amylase (AA), one maltogenic alpha-amylase (MA) and one gluco-amylase (GA) was tested. In all experiment Laminex® 750 was applied at 0.5kg/ton of corn to ensure proper filtration, with no significant impact on distribution of the fermentable sugars.

As an example of an alpha-amylase, AMYLEX® 5T (A 5T) from DuPont, was used. As example of a maltogenic alpha-amylase, DIAZYME® MA (D MA) from DuPont, was used. As an example of a gluco-amylase, DIAZYME® TGA (D TGA) from DuPont, was used. The used dosages and combinations are shown in Table 9.

TABLE 9
Concentration of enzyme product
in kg/t of corn used either as single or in combination
during mashing for wort production

| | Enzymes Applied at Mashing | | |
|---|---|---|---|
| | A 5T | D MA | D TGA |
| | kg/t of corn | | |
| 1.25 AA + 1 D MA + 0.5 D TGA | 1.25 | 1.00 | 0.5 |
| 1.25 AA + 1 D MA + 0.75 D TGA | 1.25 | 1.00 | 0.75 |
| 1.25 AA + 1 D MA + 1.0 D TGA | 1.25 | 1.00 | 1.00 |
| 1.25 AA + 0.5 D MA + 0.5 D TGA | 1.25 | 0.5 | 0.5 |
| 1.25 AA + 0.5 D MA + 0.75 D TGA | 1.25 | 0.5 | 0.75 |
| 1.25 AA + 0.5 D MA + 1.0 D TGA | 1.25 | 0.5 | 1.00 |

Wort analysis: The wort volume of each sample was measured after 30 minutes of mash separation following Dupont Standard Instruction Brewing, 23.8580-B11. In short, the sample was filtered through a plastic funnel with filter paper (VWR, European Cat. No. 516-0310, size 320 mm, folded qualitative filter paper, 307 Brewery grade, medium filtration rate) that was placed on top of a 250 ml measuring cylinder glass and time recorded. After 30 min, the amount of liquid that has passed through the filter (filtrate) into the measuring cylinder glass was measured. Original Extract (OE), Extract in the wort samples after mashing was measured using Anton Paar (Lovis) following Dupont Standard Instruction Brewing, 23.8580-B28 (Based on EBC 8.3 Extract of Wort). Fermentable sugars (% total+g/100 mL) by HPLC were DP1, DP2, DP3 and DP4+ was determined after mashing following Dupont Standard Instruction Brewing, 23.8580-B20 (Based on EBC 8.7 Fermentable Carbohydrates in Wort by HPLC (IM)). The following: wort volumes, extracts and degree of fermentables, expressed as the sum of % DP1 to DP3 released during mashing by applying the different enzymes in combinations, is shown in Table 10.

TABLE 10
Wort volume, wort extract and
relative concentration in % of the sum of DP1 to DP3.

| | Volume of | | |
|---|---|---|---|
| | Wort (mL) | | Sum of |
| | after 30 min | Extract | % DP1 to |
| 100% CORN grits | separation | (°P) | DP3 |
| 1.25 AA + 1 D MA + 0.5 D TGA | 220.0 | 14.68 | 75.09 |
| 1.25 AA + 1 D MA + 0.75 D TGA | 219.0 | 14.74 | 76.48 |
| 1.25 AA + 1 D MA + 1.0 D TGA | 218.0 | 14.75 | 77.60 |
| 1.25 AA + 0.5 D MA + 0.5 D TGA | 216.0 | 14.83 | 71.66 |
| 1.25 AA + 0.5 D MA + 0.75 D TGA | 218.0 | 14.80 | 73.29 |
| 1.25 AA + 0.5 D MA + 1.0 D TGA | 220.0 | 14.82 | 74.63 |

As seen from the amount of wort achieved (Table 10) after separation, all samples were possible to process with the applied enzymes. The extract was lowered compared to example 4 and 5 likely due to the lowered amount of (endo-acting) alpha-amylase. Notably as shown by the high percentage of DP1 to DP3 (fermentable sugars) it was possible to combine a maltogenic alpha-amylase with a gluco-amylase, besides the endo-acting alpha-amylase. These combinations of enzymes all enable good filterability, high extract and degree of fermentable sugars.

Although the foregoing invention has been described in some detail by way of illustration and example, for purposes of clarity of understanding, certain changes and modifications can be practiced within the scope of the appended claims. In addition, each reference provided herein is incorporated by reference in its entirety for all purposes to the same extent as if each reference was individually incorporated by reference. To the extent the content of any citation, including website or accession number may change with time, the version in effect at the filing date of this application is meant. Unless otherwise apparent from the context any step, element, aspect, feature of embodiment can be used in combination with any other.

1. A method of mashing for 100% adjunct brewing comprising:
a.) providing a grist comprising adjunct; and
b.) contacting the grist with an alpha amylase and a maltogenic alpha amylase and/or a glucoamylase to make a wort.

2. The method of claim 1 wherein the alpha amylase has at least 70% sequence identity to SEQ ID NO: 1.

3. The method of claim 2 wherein the alpha amylase has at least 80% sequence identity to SEQ ID NO: 1.

4. The method of claim 3 wherein the alpha amylase has at least 90% sequence identity to SEQ ID NO: 1.

5. The method of claim 4 wherein the alpha amylase has at least 95% sequence identity to SEQ ID NO: 1.

6. The method of claim 1 wherein the alpha amylase comprises an enzyme having a sequence SEQ ID NO: 1.

7. The method of claim 1 wherein the maltogenic alpha amylase has at least 70% sequence identity to SEQ ID NO: 2.

8. The method of claim 7 wherein the maltogenic alpha amylase has at least 80% sequence identity to SEQ ID NO: 2.

9. The method of claim 8 wherein the maltogenic alpha amylase has at least 90% sequence identity to SEQ ID NO: 2.

10. The method of claim 9 wherein the maltogenic alpha amylase has at least 95% sequence identity to SEQ ID NO: 2.

11. The method of claim 1 wherein the maltogenic alpha amylase comprises an enzyme having a sequence SEQ ID NO: 2.

12. The method of claim 1 wherein the glucoamylase has at least 70% sequence identity to SEQ ID NO: 3.

13. The method of claim 12 wherein the glucoamylase has at least 80% sequence identity to SEQ ID NO: 3.

14. The method of claim 13 wherein the glucoamylase has at least 90% sequence identity to SEQ ID NO: 3.

15. The method of claim 14 wherein the glucoamylase has at least 95% sequence identity to SEQ ID NO: 3.

16. The method of claim 1 wherein the glucoamylase comprises SEQ ID NO: 3.

17. The method of claim 1 wherein in step b.) the grist is contacted with an alpha amylase and a maltogenic alpha amylase.

18. The method of claim 17 wherein the alpha amylase has at least 70% sequence identity to SEQ ID NO: 1 and the maltogenic alpha amylase has at least 70% sequence identity to SEQ ID NO: 2.

19. The method of claim 18 wherein the alpha amylase has at least 80% sequence identity to SEQ ID NO: 1 and the maltogenic alpha amylase has at least 80% sequence identity to SEQ ID NO: 2.

20. The method of claim 19 wherein the alpha amylase has at least 90% sequence identity to SEQ ID NO: 1 and the maltogenic alpha amylase has at least 90% sequence identity to SEQ ID NO: 2.

21. The method of claim 20 wherein the alpha amylase has at least 95% sequence identity to SEQ ID NO: 1 and the maltogenic alpha amylase has at least 95% sequence identity to SEQ ID NO: 2.

22. The method of claim 21 wherein the alpha amylase comprises an enzyme having a sequence SEQ ID NO: 1 and the maltogenic alpha amylase comprises an enzyme having a sequence SEQ ID NO: 2.

23. The method of claim 1 wherein in step b.) the grist is contacted with an alpha amylase and a glucoamylase.

24. The method of claim 23 wherein the alpha amylase has at least 70% sequence identity to SEQ ID NO: 1 and the glucoamylase has at least 70% sequence identity to SEQ ID NO: 3.

25. The method of claim 24 wherein the alpha amylase has at least 80% sequence identity to SEQ ID NO: 1 and the glucoamylase has at least 80% sequence identity to SEQ ID NO: 3.

26. The method of claim 25 wherein the alpha amylase has at least 90% sequence identity to SEQ ID NO: 1 and the glucoamylase has at least 90% sequence identity to SEQ ID NO: 3.

27. The method of claim 26 wherein the alpha amylase has at least 95% sequence identity to SEQ ID NO: 1 and the glucoamylase has at least 95% sequence identity to SEQ ID NO: 3.

28. The method of claim 27 wherein the alpha amylase comprises an enzyme having a sequence SEQ ID NO: 1 and the glucoamylase comprises an enzyme having a sequence SEQ ID NO: 3.

29. The method of claim 1 wherein the grist is selected from the group consisting of corn, rice, sorghum and cassava or a mixture thereof.

30. The method of claim 29 wherein the grist comprises at least 10% sorghum.

31. The method of claim 30 wherein the grist comprises at least 25% sorghum.

32. The method of claim 31 wherein the grist comprises at least 50% sorghum.

33. The method of claim 32 wherein the grist comprises at least 75% sorghum.

34. The method of claim 33 wherein the grist comprises 100% sorghum.

35. The method of claim 29 wherein the grist comprises at least 10% corn.

36. The method of claim 35 wherein the grist comprises at least 25% corn.

37. The method of claim 36 wherein the grist comprises at least 50% corn.

38. The method of claim 37 wherein the grist comprises at least 75% corn.

39. The method of claim 38 wherein the grist comprises 100% corn.

40. The method of claim 29 wherein the grist comprises at least 10% rice.

41. The method of claim 40 wherein the grist comprises at least 25% rice.

42. The method of claim 41 wherein the grist comprises at least 50% rice.

43. The method of claim 42 wherein the grist comprises at least 75% rice.

44. The method of claim 43 wherein the grist comprises 100% rice.

45. The method of claim 29 wherein the grist comprises at least 10% cassava.

46. The method of claim 45 wherein the grist comprises at least 25% cassava.

47. The method of claim 46 wherein the grist comprises at least 50% cassava.

48. The method of claim 47 wherein the grist comprises at least 75% cassava.

49. The method of claim 48 wherein the grist comprises 100% cassava.

50. The method of claim 1 wherein the wort is converted to beer.

51-53. (canceled)
